(19) United States
(12) Patent Application Publication
(10) Pub. No.: US 2010/0247647 A1
(43) Pub. Date: Sep. 30, 2010

(54) SUSTAINED RELEASE TABLETS WITH HYDROMORPHONE

(75) Inventors: Marc Zingraff, Saint-Louis (FR); Markus Reher, Binningen (CH)

Correspondence Address: SMITH PATENT CONSULTING, LLC, 515 East Braddock Road, Suite B, ALEXANDRIA, VA, 22314, US

(73) Assignee: Acino Pharma AG, Liesberg (CH)

(21) Appl. No.: 12/741,963

(22) PCT Filed: Oct. 24, 2008
(86) PCT No.: PCT/EP2008/009033

§ 371 Date: May 7, 2010

(30) Foreign Application Priority Data
Nov. 9, 2007 (EP) 07021788.0

(51) Int. Cl. A61K 9/24; A61K 9/28; A61K 9/16; A61K 31/439

(52) U.S. Cl. 424/472; 424/474; 424/490; 514/282

(57) ABSTRACT

The invention relates to a tablet having a tablet core comprising a plurality of active ingredient-containing pellets and one or more pharmaceutically tolerated excipients and at least one coating applied to the tablet core, the active ingredient-containing pellets containing hydromorphone or a salt or a solvate thereof as active ingredient and having the following structure:
a) an inert core,
b) an active ingredient layer applied to the inert core,
c) a layer applied to the active ingredient layer and retarding the release of the active ingredient and
d) a further active ingredient layer on the layer retarding the release of the active ingredient.

The present invention relates to tablets having a tablet core comprising a plurality of active ingredient-containing pellets, so called “MUPS” tablets, as well as pellets which are in particular suitable for the preparation of such MUPS tablets. The drugs according to the invention contain hydromorphone as an active ingredient and are in particular distinguished in that the pellets are sustained-release pellets in which the retardation occurs via a layer applied to the active ingredient layer, but furthermore a fast-release active ingredient-containing coating is applied over this sustained-release layer.

Drugs having the active ingredient hydromorphone have been known for a long time. It is also known that hydromorphone can be administered via sustained-release formulations in which the active ingredient is released slowly over a relatively long period with a certain release profile. Such drugs are described, for example, in EP-A 271 193. This document discloses exemplary tablets in which hydromorphone hydrochloride is formulated in a retarding matrix. In general, the document also discloses that the drug can be present as a spheroid, which is provided with a film coating controlling the release. The film coating is selected so that a certain in vitro release profile is achieved. A disclosure to the effect that the pellets are compressed with customary excipients to give MUPS tablets is not made in this document.

EP-A 548 448 discloses that stability problems may frequently arise in the case of sustained-release formulations in which the active ingredient is present as a coating on an inert core when the coating is applied from an aqueous system. Formulations comprising the active ingredient hydromorphone are also mentioned as examples for such sustained-release formulations; in particular most examples in the document relate to hydromorphone. To solve these stability problems the document proposes subjecting the pellets to a particular hardening reaction. Problems which may occur in the preparation of MUPS tablets are not described in EP-A 548 448.

In the case of sustained-release formulations it is known that a fast-release constituent can be provided in order to achieve fast uptake of the active ingredient. If the sustained-release formulation is present in form of pellets, this is effected, for example, by formulating pellets which release the active ingredient rapidly together with the sustained-release pellets. It is also known that a fast-release coating comprising the active ingredient can be provided, for example over a sustained-release matrix containing the active ingredient, in order to ensure fast uptake of the active ingredient. In this regard, reference may be made, for example, to Remington: The Science and Practice of Pharmacy, 2000, page 904, and Robinson, Drugs and the Pharmaceutical Sciences, Volume 6: Sustained and Controlled Release Drug Delivery Systems, 1978, page 139.

In the case of the active ingredient hydromorphone, there are no formulations known so far in which a fast-release component is provided in addition to a sustained-release formulation. Obviously, such a fast-release ingredient is not required and/or not desired in order to achieve the release profile desired in the prior art, as described, for example, in EP-A 271 193.

If an attempt is made to formulate pellets comprising the active ingredient hydromorphone, in which an active ingredient layer is applied to an inert core and the retardation is effected via a sustained-release layer over the active ingredient layer, to give MUPS tablets, i.e. to compress them together with customary excipients and additives to give tablets, problems will arise. As described in many patents in the prior art, these methods entail the risk that the functional coating of the pellets, i.e. in the present case the sustained-release coating, will be damaged, resulting in an uncontrolled and non-reproducible change of the release profile and thus in considerable risks for the patient.

The object of the present invention is to provide pellets and MUPS tablets prepared therefrom, which do not have the abovementioned problems and in addition provide advantageous release behavior and good stability.

This object is achieved according to the invention by a MUPS tablet, i.e. a tablet having a tablet core comprising a plurality of active ingredient-containing pellets and one or more pharmaceutically tolerated excipients and at least one coating applied to the tablet core, the active ingredient-containing pellets containing hydromorphone as active ingredient and having the following structure:

a) an inert core,
b) an active ingredient layer applied to the inert core,
c) a layer applied to the active ingredient layer and retarding the release of the active ingredient and
d) a further active ingredient layer on the layer retarding the release of the active ingredient.

The invention also provides the appropriate active ingredient-containing pellets, i.e. active ingredient-containing pellets comprising the active ingredient hydromorphone, which have the following structure:

a) an inert core,
b) an active ingredient layer applied to the inert core,
c) a layer applied to the active ingredient layer and retarding the release of the active ingredient and
d) a further active ingredient layer on the layer retarding the release of the active ingredient.

The pellets according to the invention and thus also the tablets according to the invention contain hydromorphone as active ingredient. Preferably, hydromorphone is the only active ingredient present in the pellets according to the invention and the tablets according to the invention. The active ingredient is present in the tablets preferably at a concentration in the range of 0.5 to 25% by weight, in particular in the range of 0.5% by weight to 15% by weight, based on the total weight of the tablet. Preferably, a tablet according to the invention contains hydromorphone in the range of 1 to 100 mg, in particular in the range of 2 to 50 mg, more preferably in the range of 2 to 40 mg, for example in the range of 4 mg to 30 mg, most preferably in the range of 4 mg to 24 mg.

Preferably, the active ingredient is present as hydrochloride, but it can also be present as a free base, as another salt or as a solvate or as a solvate of a salt. When the term “active ingredient content” is used within the scope of this application, this always relates to the weight of the salt or solvate if a salt or solvate is employed. A solvate of the active ingredient is also understood as meaning a solvate of the salt of the active ingredient.

The pellets according to the invention have an inert core. Such inert cores are known in the prior art and are marketed, for example, as non-pareil in various sizes. The product non-pareil 18-20 (mesh) may be mentioned here as an example. In general, such inert cores have a diameter in the range of 0.2 mm to 2.5 mm, in particular in the range of 0.2 mm to 1.5 mm. They are also known under the designation “neutral cores”. Sugar cores or cores of microcrystalline cellulose are frequently used as neutral cores, but other neutral cores are also known to those skilled in the art.

According to the invention, present on the inert cores is an active ingredient layer in which the active ingredient, i.e. the hydromorphone, is applied with one or more binders as a coating on the inert core. This coating is preferably non-retarding, i.e. the hydromorphone is released rapidly from it, i.e. at least 90% within 15 minutes, determined according to the paddle-method of the U.S. Pharmacopoeia (100 rpm in 900 ml of aqueous buffer, pH in the range of 1.6 and 7.2 at 37° C.). Unless stated otherwise, all release data mentioned in this application relate to in vitro release obtained in accordance with the method of the U.S. Pharmacopoeia.

This active ingredient layer, which is present on the inert core, is referred to as “inner” active ingredient layer in this application. As a rule, the inner active ingredient layer contains a binder and the active ingredient and may, in addition to the binder and the active ingredient, also contain further customary pharmaceutically tolerated excipients and additives. Such substances are known to a person skilled in the art. Suitable binders are, for example, water-soluble polymers of low viscosity, in particular water-soluble hydroxyl-lower alkyl-celluloses, such as hydroxypropylcellulose, hydroxypropylcellulose having a low degree of substitution, hydroxypropylmethylcellulose etc. Further suitable binders are aminoalkyl methacrylate copolymers, gelatin, gum arabic, guar gum, methylcellulose, carboxymethylcellulose, ethylhydroxyethylcellulose, hydroxyethylmethylcellulose, hydroxyethylcellulose, gum tragacanth, polyvinylpyrrolidone, polyvinyl acetate, polyvinyl alcohol as well as inorganic gels, but also dextrin, sodium alginate, pectin etc.

The inner active ingredient layer too may contain, for example, colorants, plasticizers, such as triethyl citrate, polyethylene glycol, or further excipients.

Present on the inner active ingredient layer is the layer which controls the release. Such layers controlling the release of the active ingredient are known in the prior art and once again reference may be made, for example, to EP-A 271 193 or EP-A 553 392. As a rule, this layer comprises a mixture of a water-insoluble polymer and a water-soluble polymer. In principle, all water-soluble polymers which are mentioned above as binders for the inner active ingredient layer are suitable as a water-soluble polymer. For example, hydroxypropylmethylcellulose or another water-soluble cellulose, or polyvinylpyrrolidone or a similar material is particularly preferably used as a water-soluble material. As a water-insoluble polymer, for example, a wax, alone or in admixture with a fatty alcohol, water-insoluble cellulose, in particular ethylcellulose, or a polymethacrylate, for example a product of the Eudragit series, may be used. Such materials are known and, in addition to the abovementioned documents, are also described, for example, in EP-A 722 730. Moreover, mixing the water-insoluble polymer with the water-soluble polymer is effected as disclosed in the prior art. Like the inner active ingredient layer, the sustained-release coating applied to the active ingredient layer may contain further customary pharmaceutically tolerated excipients and additives, such as colorants, plasticizers, such as triethyl citrate, etc.

If the pellets are not intended to be compressed to give MUPS tablets but, for example, are filled into capsules, the pellets described above having an inert core, an inner active ingredient layer and a sustained-release coating are already usable and it is not necessary to provide a further coating. With these pellets it is already possible to achieve an advantageous release profile and advantageous release of the hydromorphone, when they are filled into capsules, sachets, etc. and administered

If the pellets are intended to be compressed to give MUPS formulations, it has however surprisingly been found according to the invention to be advantageous to provide, over the retarding coating, yet another active ingredient layer which, within the scope of this application, is referred to as “outer” active ingredient layer. In principle, the composition of the outer active ingredient layer is like the composition of the inner active ingredient layer and preferably both the outer and the inner active ingredient layers have the same constituents.

Because the pellets according to the invention also have a fast-release active ingredient-containing coating over the retarding layer, they can be easily compressed to give MUPS tablets, and the risk that compression will result in damage to the coatings such that the release profile of the pellets changes in an uncontrolled manner hereby is substantially reduced. This effect has not been described to date in the prior art for a fast-release active ingredient coating on a sustained-release coating and is surprising.

The content of hydromorphone in the inner active ingredient layer is preferably 2 mg to 80 mg, more preferably 2.4 mg to 45 mg, in particular 2.8 mg to 23.8 mg. Preferably 50% to 99% of the total content of active ingredient are present in the inner active ingredient layer, more preferably 60% to 99% of the total content of active ingredient are present in the inner active ingredient layer, and in particular 70% to 99% of the total content of active ingredient are present in the inner active ingredient layer.

Preferably 0.04 mg to 12 mg of active ingredient, more preferably 0.04 mg to 9.6 mg and in particular 0.04 mg to 7.2 mg are present in the outer active ingredient layer. The preferred percentages of the active ingredient in the outer active ingredient layer result from subtraction of the abovementioned percentages of the active ingredient in the inner active ingredient layer from 100%.

The inner active ingredient layer preferably has a thickness in the range of 10 μm to 200 μm, more preferably in the range of 10 μm to 100 μm.

The sustained-release layer preferably has a thickness in the range of 10 μm to 200 μm, more preferably in the range of 10 μm to 100 μm.

The outer active ingredient layer preferably has a thickness in the range of 10 μm to 100 μm, more preferably in the range of 10 μm to 80 μm.

All pellets preferably have a diameter in the range of 200 μm to 3000 μm, more preferably in the range of 200 μm to 2000 μm.

According to the invention, it is possible that the pellets have, in addition to the layers described, still further layers. For example, the inert core and the inner active ingredient layer or the inner active ingredient layer and the sustained-release layer, but also the sustained-release layer and the outer active ingredient layer may each also be separated by an intermediate layer. Moreover, further coatings may also be present on the outer active ingredient layer. The composition of such intermediate layers and outer coatings, respectively, is known to a person skilled in the art; for example they consist of a binder, such as, in particular, a water-soluble cellulose polymer, and optionally customary pharmaceutically acceptable excipients and additives. It is essential that these additional layers do not impair the release properties of the pellets according to the invention. However, according to the invention, the active ingredient-containing pellets preferably do not contain further layers and consist of the inert core, the inner active ingredient layer, the sustained-release layer and the outer active ingredient layer.

The sustained-release pellets according to the invention, having an inner and an outer active ingredient layer, may be compressed with customary pharmaceutically tolerated excipients to give a tablet core. The excipients for the preparation of such MUPS tablets are known to the person skilled in the art; in this context, reference may be made, for example, to the standard work of Ritschel and Bauer-Brandl, “Die Tablette”, Edition Cantor Verlag, 2002, which is hereby incorporated by reference. As a rule, fillers, binders and disintegrants, optionally also lubricants, slip agents, and mixtures thereof, are used for the preparation of the tablets. Of course, flavoring substances, colorants and further excipients can also be present. In addition to the active ingredient-containing pellets according to the invention, the tablets according to the invention preferably also contain at least one filler, more preferably at least one filler and at least one disintegrant, still more preferably at least one filler, at least one disintegrant and at least one binder. Preferably, lubricants and slip agents are also present.

Binders which may be mentioned are the same binders as those disclosed above in relation to the inner active ingredient-containing layer.

Suitable fillers are, for example, lactose, where modified lactose or anhydrous (NF) lactose may be mentioned, starch, in particular modified (pre-gelatinized) starch, native starch or mixtures of the two, calcium phosphate, in particular dibasic, unground dibasic and anhydrous dibasic calcium phosphate, cellulose derivatives, cellulose, in particular microcrystalline cellulose, mannitol, sorbitol, etc.

Of course, mixtures of different fillers can be used.

Suitable disintegrants are, for example, polyvinylpolypyrrolidone (PVPP), agar, potato starch, formaldehyde casein, sodium carboxymethylamylopectin, bentonite, sodium alginate, sodium carboxymethylcellulose, highly dispersed silica or dry pectin. As in the case of the binders and the fillers, in the case of the disintegrants too it is possible to use mixtures of different disintegrants.

Suitable flow control agents are known according to the invention; these are for example “Gleitol”, talc, colloidal silica, precipitated silica, calcium stearate, magnesium stearate, stearic acid, lauric acid, stearyl alcohol, palmitic acid, behenic acid, capric acid, carbowax or aerosil.

Suitable lubricants too are known to a person skilled in the art, and many compounds suitable as flow control agents may also be used as lubricants. Suitable lubricants are, for example, calcium stearate, behenic acid, stearic acid, aluminum stearate, stearyl alcohol, hydrogenated castor oil, palmitic acid, cetyl alcohol, talc, magnesium stearate, myristic acid, Lanette O, lauric acid, defatted milk powder, Gleitol, Talkumin, capric acid, Bolus Alba, starch and polyethylene glycols, such as carbowax 6000.

According to the invention, the cores of the MUPS tablets preferably comprise at least 10%, more preferably at least 20%, still more preferably at least 30%, in particular at least 40%, for example at least 50%, of customary excipients, the remainder being accounted for by the active ingredient-containing pellets. However, according to the invention, the active ingredient-containing pellets preferably account for at least 20%, more preferably at least 30%, of the tablet cores of the MUPS tablets.

The following table shows preferred excipients and their preferred amount in the MUPS tablet, as long as the respective excipient is employed in the tablet (remainder comprises active ingredient pellets).

| Excipient | preferred | particularly preferred | most preferred |
|---|---|---|---|
| Fillers (20 to 90%, based | lactose, cellulose, starch, | lactose, cellulose, starch, | cellulose, lactose |
| on the weight of the film | phosphate salts, mannitol, | phosphate salts | |
| tablet) | maltose, maltodextrin, | | |
| | sorbitol, sucrose | | |
| Binders (0.5 to 25%, | dextrin, dextrates, dextrose, | cellulose derivatives, | polyvinylpyrrolidone, |
| based on the weight of | cellulose derivatives, | polyvinylpyrrolidone, | cellulose derivatives |
| the film tablet) | gelatin, gums, | starch | |
| | polyvinylpyrrolidone, | | |
| | starch, sucrose | | |
| Disintegrant (1 to 25%, | PVPP, agar, bentonite, | PVPP, carboxymethylcellulose | PVPP, carboxymethyl- |
| based on the weight of | carboxymethylcellulose, | | cellulose |
| the film tablet) | sodium alginates, starch | | |
| Slip agent (0.2 to 10%, | magnesium stearate, | magnesium stearate, | magnesium stearate, |
| based on the weight of | hydrogenated castor oil, | hydrogenated castor oil, | castor oil |
| the film tablet) | glyceryl ester, polyethylene | sodium stearyl fumarate | |
| | glycol, sodium stearyl | | |
| | fumarate, stearic acid, | | |
| | talc | | |
| Flow control agent (0.1 | colloidal silica, precipitated | colloidal silica, precipitated | colloidal silica |
| to 15%, based on the | silica, starch, talc, | silica | |
| weight of the film tablet) | stearic acid, palmitic | | |
| | acid, pulverized cellulose | | |
| Colorants | FD&C and D&C blue, | FD&C and D&C blue, | titanium dioxide E 171 |
| (0.01 to 5%, based on the | green, orange, red, violet, | green, titanium dioxide | |
| weight of the film tablet) | yellow, E 100 to 180 | E 171, E 127 erythrosine, | |
| | | E 131 patent blue | |
| Other excipients | triethyl citrate, dibutyl | triethyl citrate, dibutyl | propylene glycol, triethyl |
| (0.1 to 10%, based on the | sebacate, propylene glycol, | sebacate, glyceryl monostearate, | citrate, dibutyl sebacate |
| weight of the film tablet) | diethyl phthalate, | stearic acid | |
| | dibutyl phthalate, glyceryl | | |
| | monostearate, tri- | | |
| | acetin, stearic acid | | |

The tablet cores of the MUPS tablets are provided with a customary coating as known in the prior art. The coating should not have any influence on the release of the hydromorphone; as a rule it is therefore water-soluble and is based on a water-soluble binder, as described previously in relation to the inner active ingredient layer, and customary excipients and additives. Once again, water-soluble cellulose ethers, such as HPC, HPMC, etc. and, for example, PVP are preferred as binders. The application of such coatings is known to the person skilled in the art and once again reference may be made to the abovementioned standard work “Die Tablette”.

The pellets according to the invention are preferably compressed to give MUPS tablets, but of course it is also possible to process the pellets according to the invention to give capsules, sachets or other suitable administration forms as known in the prior art.

The preparation of the pellets according to the invention is effected by methods customary in the prior art.

The preparation of the pellets is effected in 3 steps:

### 1. Active Ingredient Loading of the Cores

The various excipients and the active ingredient are dissolved/suspended in the solvent/suspending agent. The solution/suspension is sprayed onto the cores in a fluidized-bed device.

### 2. Retardation of the Active Ingredient Pellets

The various excipients are dissolved/suspended in the solvent/suspending agent. The solution/suspension is sprayed onto the active ingredient pellets in a fluidized-bed device.

3. Coating of the Sustained-Release Active Ingredient Pellets with an Additional Active Ingredient Layer

The various excipients and the remaining active ingredient are dissolved/suspended in the solvent/suspending agent. The solution/suspension is applied onto the sustained-release pellets in a fluidized-bed device.

The compression of the pellets according to the invention to give MUPS tablets is also effected in a manner known in the prior art, for example as follows.

The finished pellets are mixed with other excipients in a suitable mixer until the mixture is homogenous. The mixing times as well as the particle size distribution of the various excipients, in particular of the fillers, are suitably adjusted by a person skilled in the art.

The so-called final mixture is then tabletted on a tablet press. The tabletting rate and tabletting pressure of the tablet cores are suitably adjusted by a person skilled in the art.

The tablet cores are then coated with a non-functional lacquer. The various excipients are dissolved/suspended in the solvent/suspending agent. The solution/suspension is sprayed onto the tablet cores in a suitable device (either air coater or drum coater).

The release profile of hydromorphone from the pellets according to the invention and MUPS tablets, respectively, is as described in the prior art for the known hydromorphone formulations and, in this respect, reference may be made in particular to EP-A 548 448 and EP-A 271 193, the content of disclosure of which is hereby incorporated by reference.

The following examples explain the invention.

### Example for the Preparation of Pellets:

1. Polyethylene glycol is dissolved with hydroxypropylmethylcellulose and hydromorphone HCl in water. Talc is suspended separately in water and then added to the hydromorphone solution. The resulting suspension is sprayed onto sugar pellets at a product temperature of 39-45° C. in a Glatt fluidized-bed device.
2. Ethylcellulose is dissolved together with propylene glycol and hydroxypropylcellulose in ethanol. In addition, talc can be suspended separately in water or ethanol and added to the ethylcellulose solution. The resulting solution/suspension is sprayed onto the hydromorphone HCl active ingredient pellets at a product temperature of 39-50° C. in the Glatt fluidized-bed device.
3. Polyethylene glycol is dissolved with hydroxypropylmethylcellulose and hydromorphone HCl in water. Talc is suspended separately in water and then added to the hydromorphone solution. The resulting suspension is sprayed onto sustained-release pellets at a product temperature of 39-45° C. in the Glatt fluidized-bed device.

### Example for the Preparation of MUPS Tablets:

1. The finished pellets are mixed with microcrystalline cellulose and screened colloidal silica. Subsequently, screened magnesium stearate is added and the mixture is further mixed.
2. The final mixture is tabletted. The tabletting rate is adjusted such that the final mixture remains homogenous on tabletting. The tabletting pressure is suitably adjusted.
3. The coating suspension is prepared as follows. Hydroxypropylmethylcellulose is dissolved with polyethylene glycol in water. Talc is suspended separately with titanium dioxide in water and then added to the hydroxypropylmethylcellulose solution. The resulting suspension is sprayed onto the tablet cores at a product temperature of 39-45° C. in a Glatt Coater.

1. A tablet comprised of a tablet core having at least one coating applied thereto, said tablet core comprising a plurality of active ingredient-containing pellets and one or more pharmaceutically tolerated excipients, the active ingredient-containing pellets containing hydromorphone or a salt or a solvate thereof as active ingredient and further comprising the following structure:
a) an inert core,
b) an active ingredient layer applied to the inert core,
c) a layer applied to the active ingredient layer and retarding the release of the active ingredient and
d) a further active ingredient layer on the layer retarding the release of the active ingredient.

2. The tablet according to claim 1, wherein said tablet contains the active ingredient at a concentration in the range of 2 to 30% by weight, based on the total weight of the tablet.

3. The tablet according to claim 1, wherein the pharmaceutically tolerated excipients are pressed with the active ingredient-containing pellets to yield the tablet core, further wherein said excipients are selected from the group consisting of binders, fillers, disintegrants, lubricants, slip agents, and mixtures thereof.

4. The tablet according to claim 1, wherein said at least one coating applied to the tablet core is not a layer retarding the release of the active ingredient.

5. An active ingredient-containing pellet comprising the active ingredient hydromorphone and having following structure:
a) an inert core,
b) an active ingredient layer applied to the inert core,
c) a layer applied to the active ingredient layer and retarding the release of the active ingredient and
d) a further active ingredient layer on the layer retarding the release of the active ingredient.

6. The active ingredient-containing pellet according to claim 5, characterized in that the layer c) applied to the active ingredient layer b) and retarding the release of the active ingredient contains ethylcellulose as a retarding agent.

7. The active ingredient-containing pellet according to claim 5, characterized in that the content of hydromorphone in the active ingredient layer c) applied to the inert core a) is in the range of 2 mg to 80 mg.

8. The active ingredient-containing pellet according to claim 5, characterized in that the content of hydromorphone in the further active ingredient layer d) applied to the layer c) retarding the release of the active ingredient is in the range of 0.04 mg to 12 mg.

9. The active ingredient-containing pellet according to claim 5, characterized in that the active ingredient layer b) applied to the inert core a) has a thickness in the range of 10 μm to 200 μm.

10. The active ingredient-containing pellet according to claim 5, characterized in that the further active ingredient layer d) applied to the layer c) retarding the release of the active ingredient has a thickness in the range of 10 μm to 200 μm.
